(19) United States
(12) Patent Application Publication
(10) Pub. No.: US 2016/0202204 A1
(43) Pub. Date: Jul. 14, 2016

(54) ELECTROCHEMICAL-BASED ANALYTICAL TEST STRIP WITH ULTRA-THIN DISCONTINUOUS METAL LAYER

(71) Applicant: CILAG GmbH International, Zug (CH)

(72) Inventors: James RODGERS, Inverness (GB); Alexander COOPER, Inverness (GB); Gavin MACFIE, Inverness (GB); Steven SETFORD, Inverness (GB); Russell BAIN, Inverness (GB); Scott SLOSS, Inverness (GB)

(73) Assignee: Cilag GmbH International

(21) Appl. No.: 14/917,080

(22) PCT Filed: Sep. 10, 2014
(86) PCT No.: PCT/EP2014/069323

§ 371 Date: Mar. 7, 2016

(30) Foreign Application Priority Data
Sep. 11, 2013 (GB) 1316193.0

(51) Int. Cl. G01N 27/327; G01N 33/487; C12Q 1/00; G01N 27/30; C23C 14/18

(52) U.S. Cl. CPC G01N 27/3272; G01N 27/308; C23C 14/185; C12Q 1/006; G01N 33/48707

(57) ABSTRACT

An electrochemical-based analytical test strip for the determination of an analyte (such as glucose) in a bodily fluid sample includes an electrically insulating base layer, a first electrically conductive layer disposed on the electrically insulating base layer and including at least one electrode, an enzymatic reagent layer disposed on the at least one electrode, a patterned spacer layer and a top layer. The electrochemical-based analytical test strip also includes an ultra-thin discontinuous metal layer with a nominal thickness of less than 10 nanometers disposed between the first electrically conductive layer and the top layer. Moreover, at least the patterned spacer layer defines a sample-receiving chamber containing the at least one electrode, and the ultra-thin discontinuous metal layer is disposed at least within the sample-receiving chamber.

### FIELD OF THE INVENTION

The present invention relates, in general, to medical devices and, in particular, to analytical test strips and related methods.

### BACKGROUND OF THE INVENTION

The determination (e.g., detection and/or concentration measurement) of an analyte in, or a characteristic of, a fluid sample is of particular interest in the medical field. For example, it can be desirable to determine glucose, ketone bodies, cholesterol, lipoproteins, triglycerides, acetaminophen, hematocrit and/or HbA1c concentrations in a sample of a bodily fluid such as urine, blood, plasma or interstitial fluid. Such determinations can be achieved using analytical test strips, based on, for example, visual, photometric or electrochemical techniques. Conventional electrochemical-based analytical test strips are described in, for example, U.S. Pat. Nos. 5,708,247 and 6,284,125, each of which is hereby incorporated in full by reference.

### SUMMARY OF INVENTION

In a first aspect, there is provided an electrochemical-based analytical test strip for the determination of an analyte in a bodily fluid sample, the electrochemical-based analytical test strip comprising:

an electrically insulating base layer;

a first electrically conductive layer disposed on the electrically insulating base layer and including at least one electrode;

an enzymatic reagent layer disposed on the at least one electrode;

a patterned spacer layer;

a top layer; and

an ultra-thin discontinuous metal layer with a nominal thickness of less than 10 nanometers disposed between the first electrically conductive layer and the top layer,

wherein at least the patterned spacer layer defines a sample-receiving chamber containing the at least one electrode, and

wherein the ultra-thin discontinuous metal layer may be disposed at least within the sample-receiving chamber.

The first electrically conductive layer may be a carbon electrically conductive layer.

The ultra-thin discontinuous metal layer may be an ultra-thin discontinuous gold layer.

The at least one electrode may be a plurality of electrodes and the ultra-thin discontinuous metal layer may be disposed on the electrically-insulating base layer and the first electrically conductive layer including at least one of the plurality of electrodes.

The ultra-thin discontinuous metal layer may be disposed on the plurality of electrodes.

The plurality of electrodes may include a working electrode and a counter electrode and with respect to the plurality of electrodes, the ultra-thin discontinuous layer may be disposed only on the counter electrode.

The discontinuous nature of the ultra-thin discontinuous metal layer may be predetermined such as to preclude an electrical path between the plurality of electrodes via the ultra-thin discontinuous metal layer.

The electrochemical-based analytical test strip may further include:

a second electrically conductive layer disposed immediately below the top layer and including at least one electrode disposed in the sample-receiving chamber,

wherein the ultra-thin discontinuous metal layer may be disposed on the second electrically conductive layer.

The second electrically conductive layer may include polymer-bound graphite particles and may be of free-standing mechanical integrity.

The bodily fluid sample may be a whole blood sample and the analyte may be glucose.

The nominal thickness of the ultra-thin discontinuous metal layer may be in the range of 1 nano-meter to 4 nano-meters.

The ultra-thin discontinuous metal layer may have discontinuities in the range of 5 discontinuities per micron to 20 discontinuities per micron.

The ultra-thin discontinuous metal layer may be a sputter-deposited ultra-thin discontinuous metal layer.

The sputter-deposited ultra-thin discontinuous metal layer may be a sputter-deposited ultra-thin discontinuous gold layer.

The sputter-deposited ultra-thin discontinuous metal layer may be a sputter-deposited ultra-thin discontinuous metal layer may be formed of at least one of palladium, platinum and silver.

The ultra-thin discontinuous metal layer may include metal islands with a diameter no greater than 100 microns.

In a second aspect, there is provided a method comprising:

introducing a bodily fluid sample into a sample-receiving chamber of an electrochemical-based analytical test strip, the electrochemical-based analytical test strip including:

an electrically insulating base layer;

at least one electrode disposed within the sample-receiving chamber and on the electrically-insulating base layer; and

an ultra-thin discontinuous metal layer with a nominal thickness of less than 10 nanometers disposed above the at least one electrode and at least within the sample-receiving chamber;

detecting an electrochemical response of the at least one electrode of the electrochemical-based analytical test strip; and

determining an analyte in the bodily fluid sample based on the detected electrochemical response.

The at least one electrode may be a carbon electrode.

The ultra-thin discontinuous metal layer may be an ultra-thin discontinuous gold layer.

The at least one electrode may be a plurality of electrodes and the ultra-thin discontinuous metal layer may be disposed on the electrically-insulating base layer and the first electrically conductive layer including at least one of the plurality of electrodes.

The ultra-thin discontinuous metal layer may be disposed on the plurality of electrodes.

The plurality of electrodes may include a working electrode and a counter electrode and, with respect to the plurality of electrodes, the ultra-thin discontinuous layer may be disposed only on the counter electrode.

The discontinuous nature of the ultra-thin discontinuous metal layer may be predetermined such as to preclude an electrical path between the plurality of electrodes via the ultra-thin discontinuous metal layer.

The bodily fluid sample may be a whole blood sample and the analyte may be glucose.

The nominal thickness of the ultra-thin discontinuous metal layer may be in the range of 1 nano-meter to 4 nano-meters.

The ultra-thin discontinuous metal layer may have discontinuities in the range of 5 discontinuities per micron to 20 discontinuities per micron.

The ultra-thin discontinuous metal layer may be a sputter-deposited ultra-thin discontinuous metal layer.

The sputter-deposited ultra-thin discontinuous metal layer may be a sputter-deposited ultra-thin discontinuous gold layer.

The sputter-deposited ultra-thin discontinuous gold layer may include gold islands with a diameter no greater than 100 microns.

### BRIEF DESCRIPTION OF DRAWINGS

The accompanying drawings, which are incorporated herein and constitute part of this specification, illustrate presently preferred embodiments of the invention, and, together with the general description given above and the detailed description given below, serve to explain features of the invention, in which:

FIG. 1A is a simplified exploded perspective view of an electrochemical-based analytical test strip according to an embodiment of the present invention;

FIG. 1B is a simplified exploded perspective view of an electrochemical-based analytical test strip according to an alternative embodiment of the present invention;

FIG. 2 is a simplified perspective view of the electrochemical-based analytical test strip of FIG. 1A;

FIG. 3 is a simplified cross-sectional side view (not to scale) of a portion of the electrochemical-based analytical test strip of FIG. 1 taken along line A-A of FIG. 2;

FIG. 4 is a simplified cross-sectional end view (not to scale) of a portion of the electrochemical-based analytical test strip of FIG. 1 taken along line B-B of FIG. 2;

FIG. 5 is a graphical depiction of reciprocal resistance versus nominal deposition thickness for gold (Au) metal layers prepared using conventional sputtering techniques;

FIG. 6 is a graphical depiction of electrochemical responses produced using cyclic voltammetry for conventional electrochemical-based analytical test strips (labeled “standard) and electrochemical-based analytical test strips according to an embodiment of the present invention (labeled “sputtered”);

FIG. 7 is a graphical depiction of electrochemical responses produced using cyclic voltammetry for a conventional electrochemical-based analytical test strips (labeled “control”) and a set of electrochemical-based analytical test strips according to embodiments of the present invention with ultra-thin discontinuous gold layers with nominal thicknesses in the range of 2 nm to 6 nm;

FIG. 8 is a simplified exploded perspective depiction of an electrochemical-based analytical test strip according to another embodiment of the present invention;

FIG. 9 is a simplified side view depiction of a portion of the electrochemical-based analytical test strip of FIG. 8 that also depicts electrical connection to an associated hand-held test meter (not entirely shown) via electrical connections EC of the hand-held test meter; and

FIG. 10 is a flow diagram depicting stages in a method for determining an analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

Also as used herein, the term “nominal thickness” refers to a thickness that is determined based on the amount of metal deposited over a relatively large area and the assumption of a continuous uniform film and, therefore, may not represent the actual thickness of any given portion of the ultra-thin discontinuous metal layer. For example, an ultra-thin discontinuous metal layer with a nominal thickness of 5 nano-meters includes islands of metal (also referred to as metal islands) with actual thicknesses of greater than 5 nano-meters separated by areas of no metal (i.e., “bare” regions with an actual metal thickness of zero or essentially zero).

In addition, as used herein, the term “discontinuous” refers to a layer with breaks (i.e., discontinuities) in the layer structure sufficient to prevent electrical bridging when the ultra-thin discontinuous metal layer is disposed across neighboring but spaced apart electrodes and an underlying electrically-insulating base layer. The density of such discontinuities can be, for example, in the range of 5 discontinuities per micron to 20 discontinuities per micron and the diameter of the metal islands can beneficially be, for example, no greater than 100 microns. Such a discontinuity range (i.e., 5 to 20 discontinuities per micron as measured by cross-section across an area where an ultra-thin discontinuous gold metal layer has been deposited by sputtering) provided an unexpectedly beneficial enhancement in electrochemical response of the electrochemical-based analytical test strip without creating an electrically short circuit from electrode to electrode across an electrically-insulating base layer).

An electrochemical-based analytical test strip for the determination of an analyte (such as glucose) in a bodily fluid sample (such as, for example, a whole blood sample) includes an electrically insulating base layer, a first electrically conductive layer disposed on the electrically insulating base layer and including at least one electrode, an enzymatic reagent layer disposed on the at least one electrode, a patterned spacer layer and a top layer. The electrochemical-based analytical test strip also includes an ultra-thin discontinuous metal layer with a nominal thickness of less than 10 nanometers disposed between the first electrically conductive layer and the top layer. Moreover, at least the patterned spacer layer defines a sample-receiving chamber containing the at least one electrode, and the ultra-thin discontinuous metal layer is disposed at least within the sample-receiving chamber.

The at least one electrode can, for example, be a plurality of electrodes and the ultra-thin discontinuous metal layer can be, for example, disposed on the first electrically insulating base layer and the first electrically-conductive layer (including at least one of the plurality of electrodes) but under the enzymatic reagent layer. Alternatively, electrochemical-based analytical test strips according to embodiments of the present invention can include a second electrode disposed immediately under the top layer and at least partially in the sample-receiving chamber and the ultra-thin discontinuous metal layer can be disposed on such a second layer.

Electrochemical-based analytical test strips according to embodiments of the present invention are beneficial in that, for example, the ultra-thin discontinuous metal layer can provide a beneficial improvement in electrochemical response of the electrochemical-based analytical test strips in comparison to electrochemical-based analytical test strips devoid of such an ultra-thin discontinuous metal layer. The enhanced electrochemical response can, for example, enable a reduction in the sample-receiving chamber volume and, hence, a reduction in bodily fluid sample size. In addition, the ultra-thin nature of the ultra-thin discontinuous metal layers (i.e., less than 10 nm in nominal thickness) results in reduced usage of the metal (e.g., gold) in the ultra-thin discontinuous metal layer, thus saving cost. Moreover, the discontinuous nature of the ultra-thin discontinuous metal layer prevents the ultra-thin metal layer form serving as an electrical short circuit when, for example, the ultra-thin discontinuous metal layer is disposed on a plurality of electrodes and across an electrically-insulating baser layer on which the plurality of electrodes is disposed. In other words, the discontinuities essentially eliminate any metal bridging between the plurality of electrodes by the ultra-thin discontinuous metal layer.

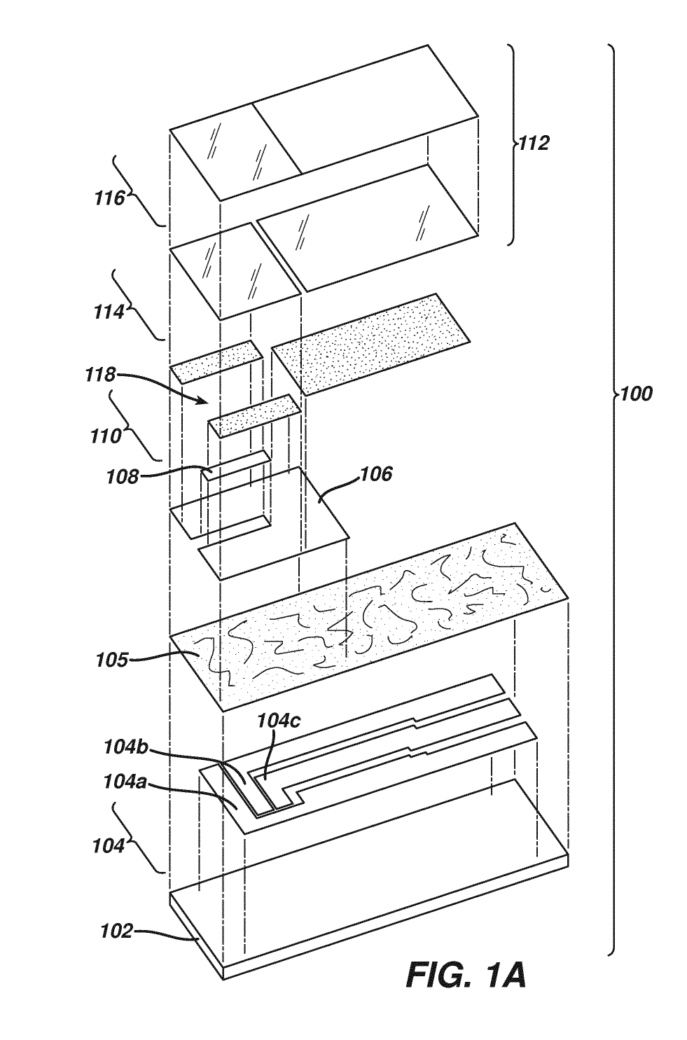

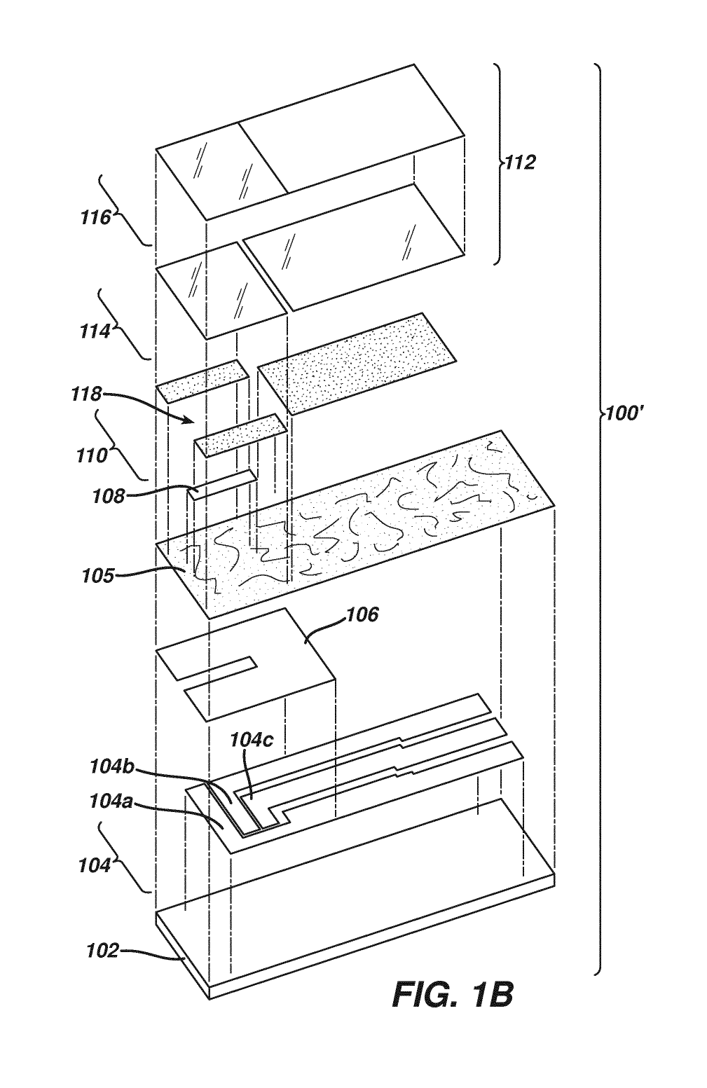

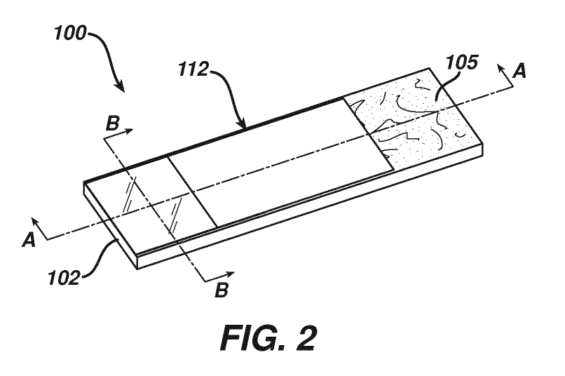

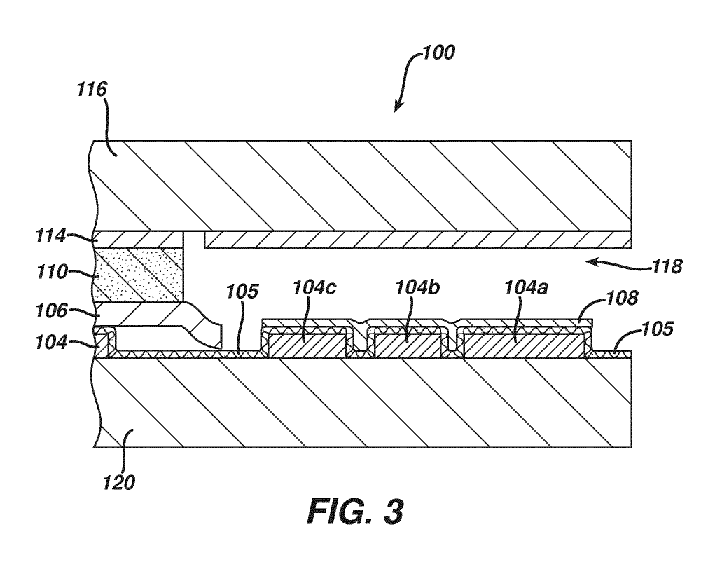

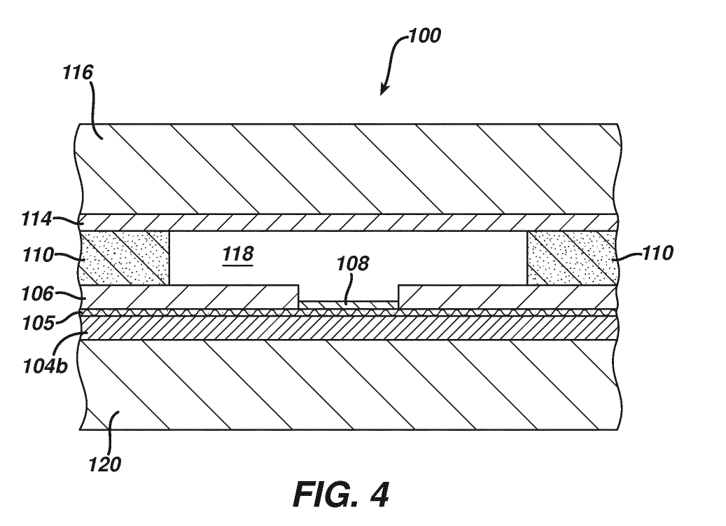

FIG. 1A is a simplified exploded perspective view of an electrochemical-based analytical test strip 100 according to an embodiment of the present invention. FIG. 2 is a simplified perspective view of electrochemical-based analytical test strip 100. FIG. 3 is a simplified cross-sectional side view (not to scale) of a portion of electrochemical-based analytical 100 taken along line A-A of FIG. 2. FIG. 4 is a simplified cross-sectional end view (also not to scale) of a portion of the electrochemical-based analytical test strip 100 taken along line B-B of FIG. 2.

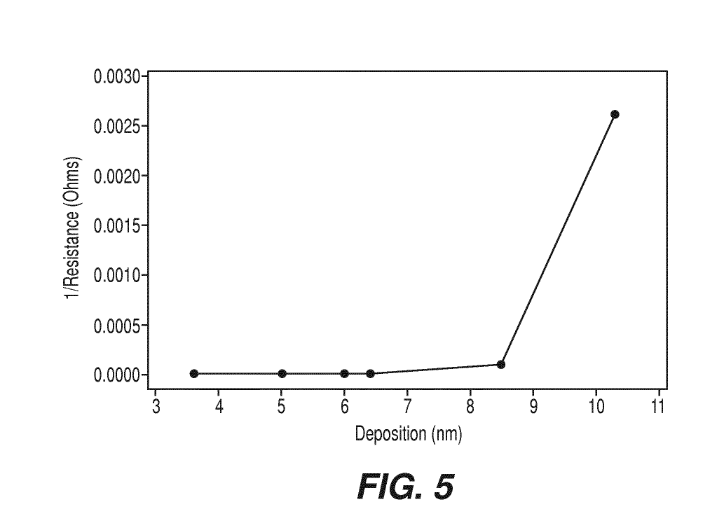

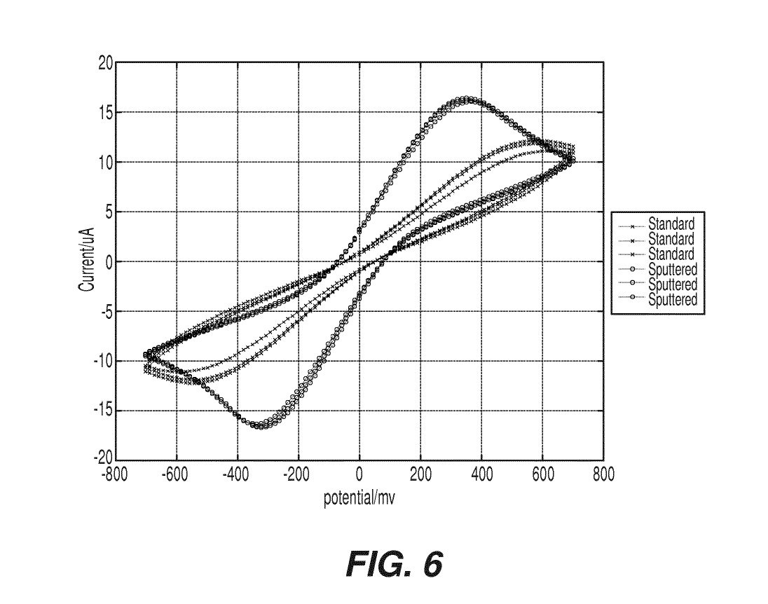

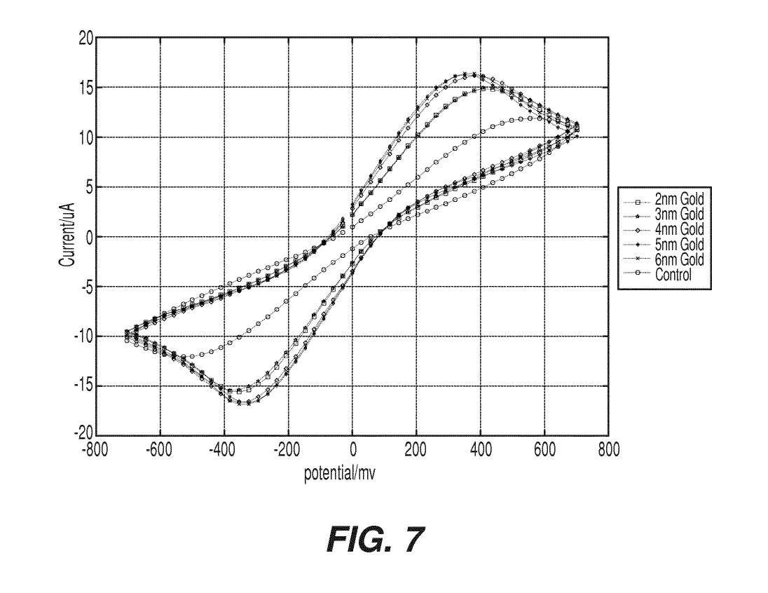

FIG. 5 is a graphical depiction of reciprocal resistance versus nominal deposition thickness for gold (Au) metal layers prepared using conventional sputtering techniques. FIG. 6 is a graphical depiction of electrochemical responses produced using cyclic voltammetry for conventional electrochemical-based analytical test strips (labeled “standard) and electrochemical-based analytical test strips according to an embodiment of the present invention (labeled “sputtered”). FIG. 7 is a graphical depiction of electrochemical responses produced using cyclic voltammetry for a conventional electrochemical-based analytical test strips (labeled “control”) and a set of electrochemical-based analytical test strips according to embodiments of the present invention with ultra-thin discontinuous gold layers with nominal thicknesses in the range of 2 nm to 6 nm.

Referring to FIGS. 1A and 2 through 7, electrochemical-based analytical test strip 100 for the determination of an analyte (such as glucose) in a bodily fluid sample (for example, a whole blood sample) includes an electrically-insulating base layer 102, a patterned electrically conductive layer 104, an ultra-thin discontinuous metal layer 105, a patterned insulation layer 106, an enzymatic reagent layer 108, a patterned spacer layer 110, and a top layer 112 consisting of a hydrophilic sub-layer 114 and a top tape 116.

In the embodiment of FIGS. 1A and 2 through 4, at least the patterned spacer layer and top layer define a sample-receiving chamber 118 within electrochemical-based analytical test strip 100 (see FIGS. 3 and 4 in particular).

Electrically-insulating base layer 102 can be any suitable electrically-insulating base layer known to one skilled in the art including, for example, a nylon base layer, a polycarbonate base layer, a polyimide base layer, a polyvinyl chloride base layer, a polyethylene base layer, a polypropylene base layer, a glycolated polyester (PETG) base layer, or a polyester base layer. The electrically-insulating base layer can have any suitable dimensions including, for example, a width dimension of about 5 mm, a length dimension of about 27 mm and a thickness dimension of about 0.5 mm.

Electrically-insulating base layer 102 provides structure to electrochemical-based analytical test strip 100 for ease of handling and also serves as a base for the application (e.g., printing or deposition) of subsequent layers (e.g., a patterned electrically conductor layer and an ultra-thin discontinuous metal layer).

Patterned electrically conductive layer 104 is disposed on the electrically-insulating base layer 102 and includes a first electrode 104a, a second electrode 104b and a third electrode 104c. First electrode 104a, second electrode 104b and third electrode 104c can be, for example, configured as a counter/reference electrode, a first working electrode and a second working electrode, respectively. Therefore, the second and third electrodes are also referred to herein as working electrodes 104b and 104c and the first electrode as counter electrode 104a. Although, for the purpose of explanation only, electrochemical-based analytical test strip 100 is depicted as including a total of three electrodes, embodiments of electrochemical-based analytical test strips, including embodiments of the present invention, can include any suitable number of electrodes.

Patterned electrically conductive layer 104, including first electrode 104a, second electrode 104b and third electrode 104c, of electrochemical-based analytical test strip 100 can be formed of any suitable conductive material including, for example, electrically conducting carbon-based materials including carbon inks. It should be noted that patterned electrically conductive layers employed in electrochemical-based analytical test strips according to embodiments of the present invention can take any suitable shape and be formed of any suitable materials including, for example, metal materials and conductive carbon materials.

Referring in particular to FIGS. 1A, 3 and 4, the disposition of first electrode 104a, second electrode 104b and third electrode 104c and enzymatic reagent layer 108 are such that electrochemical-based analytical test strip 100 is configured for the electrochemical determination of an analyte (such as glucose) in a bodily fluid sample (such as a whole blood sample) that has filled sample-receiving chamber 118.

Ultra-thin discontinuous metal layer 105 has a nominal thickness of less than 10 nano-meters and preferably in the range of 1 nano-meter to 5 nano-meters. Ultra-thin discontinuous metal layer 105 can be formed of any suitable metal including, but not limited to, for example, gold (Au), silver (Ag), platinum (Pt) and palladium (Pd). As described herein with, for example, respect to FIGS. 5, 6 and 7, the combination of an ultra-thin discontinuous gold layer and a carbon electrode is particularly beneficial in that it provides enhanced electrochemical responses at low cost and precludes shorting between adjacent electrodes.

In the embodiment of FIGS. 1A, 3 and 4 (and also the embodiment of FIG. 1B described below), ultra-thin discontinuous metal layer 105 is disposed on all of the plurality of electrodes 104a, 104b and 104c. However, if desired, ultra-thin discontinuous metal layer 105 can be disposed on only the counter electrode 104a and not on first working electrode 104b and second working electrode 104c. In this manner, the electrochemical reactivity of counter electrode 104a can be enhanced (relative to working electrodes 104b and 104c), thus enabling use of a counter electrode of beneficially reduced area while providing an electrochemical response equivalent to a larger counter electrode devoid of the ultra-thin discontinuous metal layer. A counter electrode of reduced area in turn enables use of a sample-receiving chamber of beneficially reduced volume.

As also described elsewhere in this disclosure, the discontinuous nature of ultra-thin discontinuous metal layer 105 is predetermined such as to preclude a deleterious electrical path between electrodes 104a, 104b and 104c via the ultra-thin discontinuous metal layer. For example, the ultra-thin discontinuous metal layer can have discontinuities in the range of 5 discontinuities per micron to 20 discontinuities per micron (as measured by cross-section in the perspective of FIG. 3 or FIG. 4. Such a range of discontinuities can be readily manufactured by sputter depositing an ultra-thin discontinuous metal layer (such as a sputtered gold (Au) metal layer) at a nominal thickness of less than 10 nano-meters and, preferably, at a nominal thickness in the range of 1 nano-meter to 4 nano-meters.

Enzymatic reagent layer 108 is disposed on at least a portion of patterned electrically conductor layer 104. Enzymatic reagent layer 108 can include any suitable enzymatic reagents, with the selection of enzymatic reagents being dependent on the analyte to be determined. For example, if glucose is to be determined in a blood sample, enzymatic reagent layer 108 can include a glucose oxidase or glucose dehydrogenase along with other components necessary for functional operation. Enzymatic reagent layer 108 can include, for example, glucose oxidase, tri-sodium citrate, citric acid, polyvinyl alcohol, hydroxyl ethyl cellulose, potassium ferricyanide, potassium ferrocyanide, antifoam, fumed silica (either with or without a hydrophobic surface modification), PVPVA, and water. Further details regarding reagent layers, and electrochemical-based analytical test strips in general, are in U.S. Pat. Nos. 6,241,862 and 6,733,655, the contents of which are hereby fully incorporated by reference.

Patterned insulation layer 106 can be formed of any suitable electrically-insulating dielectric material including commercially available screen-printable dielectric inks.

Patterned spacer layer 110 can be formed, for example, from a screen-printable pressure sensitive adhesive commercially available from Apollo Adhesives, Tamworth, Staffordshire, UK. In the embodiment of FIGS. 1A, 1B, 2, 3 and 4, patterned spacer layer 110 defines outer walls of the sample-receiving chamber 118. Patterned spacer layer 110 can have a thickness of, for example, approximately 110 microns, be electrically nonconductive, and be formed of a polyester material with top and bottom side acrylic-based pressure sensitive adhesive.

Top layer 112 can be, for example, a clear film with hydrophilic properties that promote wetting and filling of electrochemical-based analytical test strip 100 by a fluid sample (e.g., a whole blood sample). Such clear films are commercially available from, for example, 3M of Minneapolis, Minn. U.S.A. and Coveme (San Lazzaro di Savena, Italy). Top layer 112 can be, for example, a polyester film coated with a surfactant that provides a hydrophilic contact angle <10 degrees. Top layer 112 can also be a polypropylene film coated with a surfactant or other surface treatment. In such a circumstance, the surfactant coating serves as hydrophilic sub-layer 114. Top layer 112 can have a thickness, for example, of approximately 100 μm.

Electrochemical-based analytical test strip 100 can be manufactured, for example, by the sequential aligned formation of patterned electrically conductive layer 104, ultra-thin discontinuous metal layer 105, patterned insulating layer 106, enzymatic reagent layer 108, patterned spacer layer 110 and top layer 112. Any suitable techniques known to one skilled in the art can be used to accomplish such sequential aligned formation, including, for example, screen printing, photolithography, photogravure, chemical vapour deposition and tape lamination techniques. However, as described herein, ultra-thin discontinuous metal layers employed in embodiments of the present invention can be readily deposited using conventional metal sputtering techniques that result in the deposition of discontinuous layers at thicknesses of less than approximately 10 nm.

FIG. 5 (a graphical depiction of reciprocal resistance versus nominal deposition thickness for gold (Au) metal layers prepared using conventional sputtering techniques) depicts the reciprocal resistance between a pair of neighboring, spaced-apart carbon electrodes with discontinuous gold layers deposited thereon and spaced apart by a minimum distance of 200 microns. Such neighboring spaced-apart electrodes are also referred to as “adjacent” electrodes although they are not contacting one another. FIG. 5 illustrates that for deposited gold layers with a nominal thickness of less than 10 nanometers, the resistance is very high, thus indicating that ultra-thin discontinuous gold layers of less than 10 nano-meters in nominal thickness are precluding electrical short circuits between the adjacent electrodes.

Referring to FIGS. 6 and 7, electrochemical-based analytical test strips according to an embodiment of the present invention that included carbon electrodes and a sputter-deposited ultra-thin discontinuous gold (Au) layers displayed enhanced electrochemical response in comparison to control electrochemical-based analytical test strips that includes carbon electrodes but no ultra-thin discontinuous metal layers (see FIGS. 6 and 7). Moreover, the enhanced electrochemical response was present at nominal thicknesses of 2 nano-meters, 3 nano-meters, 4 nano-meters and 6 nano-meters, which was unexpected given that the films are discontinuous. in nature.

The data of FIGS. 6 and 7 was obtained using a potentiostat and by filling the electrochemical-based analytical test strips with a solution of 20 mM Ferricyanide, 20 mM Ferrocyanide and 1M Potassium Chloride. The potentiostat applied a potential with a scan range of −0.7 to +0.7V and a scan rate of 50 mV/second.

FIG. 1B is a simplified exploded perspective view of an electrochemical-based analytical test strip 100′ according to an alternative embodiment of the present invention in which like labeling numerals indicate like elements from FIG. 1. Electrochemical-based analytical test strip 100′ is identical to electrochemical-based analytical test strip 100 with the exception that ultra-thin discontinuous metal layer 105 is disposed above patterned insulation layer 106 instead of below patterned insulation layer 106 as in FIG. 1. However, the configuration (i.e., “pattern”) of patterned insulation layer 106 is such that ultra-thin-discontinuous metal layer 105 is disposed on the electrodes of patterned conductor layer 104. Electrochemical-based analytical test strip 100′, therefore, represents an alternative configuration to electrochemical-based analytical test strip 100. However, in both electrochemical-based analytical test strip 100 and electrochemical-based analytical test strip 100′, ultra-thin discontinuous metal layer 105 is disposed above the electrode(s) of the patterned electrically conductive layer.

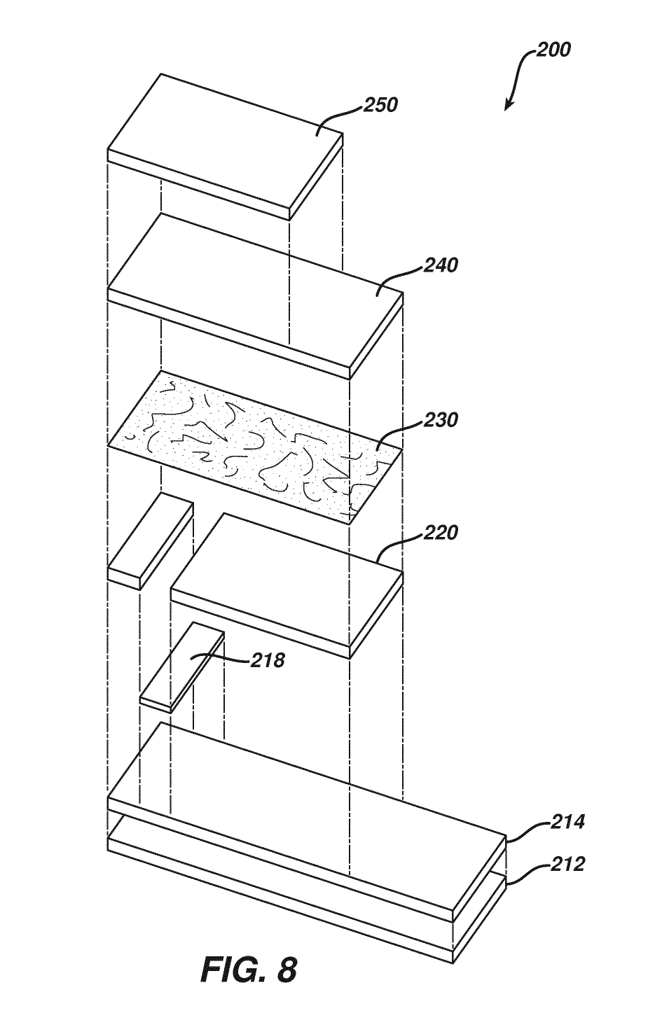

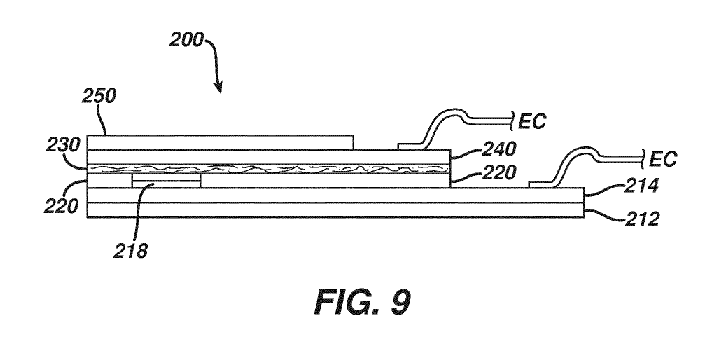

FIG. 8 is a simplified exploded perspective depiction of an electrochemical-based analytical test strip 200 according to another embodiment of the present invention. FIG. 9 is a simplified, side view depiction of a portion of the electrochemical-based analytical test strip 200 that also depicts electrical connection to an associated hand-held test meter (not entirely shown) via electrical connections EC of the hand-held test meter.

Referring to FIGS. 8 and 9, electrochemical-based analytical test strip 200 includes an electrically insulating base layer 212, a first electrically conductive layer 214 disposed on electrically insulating base layer 212 and including first electrode 214a, and an enzymatic reagent layer 218 disposed on first electrode 214a. Electrochemical-based analytical test strip 200 also includes a patterned spacer layer 220, an ultra-thin discontinuous metal layer 230, a second electrically conductive layer 240 including second electrode 240a and a top layer.

In the embodiment of FIGS. 8 and 9, ultra-thin discontinuous metal layer 230 has a nominal thickness of less than 10 nanometers. In addition, patterned spacer layer 220 defines a sample-receiving chamber 250 containing first electrode 214a and 240a in a co-facial (opposing) configuration.

Ultra-thin discontinuous layer 230 is beneficial in that the electrochemical responses of electrochemical-based analytical test strip 200 are enhanced, thus enabling the use of a second electrically conductive layer 240 that is formed of polymer-bound graphite particles and that is of a free-standing mechanical integrity. Such free-standing layers (i.e., structurally free-standing layers formed of polymer-bound graphite particles), although electrically conductive, have electrochemical properties that are not particularly suitable or optimized for use in electrochemical-based analytical test strips in the absence of the ultra-thin discontinuous metal layers described herein or the presence of more expensive thick continuous metal layers. However, employing ultra-thin discontinuous metal layers (e.g., ultra-thin discontinuous gold layers) in combination with such free-standing electrically conductive layers provides for structural rigidity and a suitable electrochemical response.

The structural rigidity of the free-standing second electrically conductive layer 240 enables manufacturing of the configuration depicted in FIGS. 8 and 9 wherein operable contact by an electrical connector to second electrically conductive layer 240 and first electrically conductive layer 214 occurs in a non-opposing configuration.

Second electrically-conductive layer 240 can be formed of any suitable material including, for example, free-standing polymer-bound graphite materials commercially available from Adhesive Research as part number MH95000 or commercially available from, Exopack (Wrexham Scotland) as Vinyl 2267 and Vinyl 2252 under the “Inspire Medical” brand. The remainder of the layers of electrochemical-based analytical test strip 200 can be formed of the same materials as described with respect to the layers of electrochemical-based analytical test strip 100 performing the corresponding function.

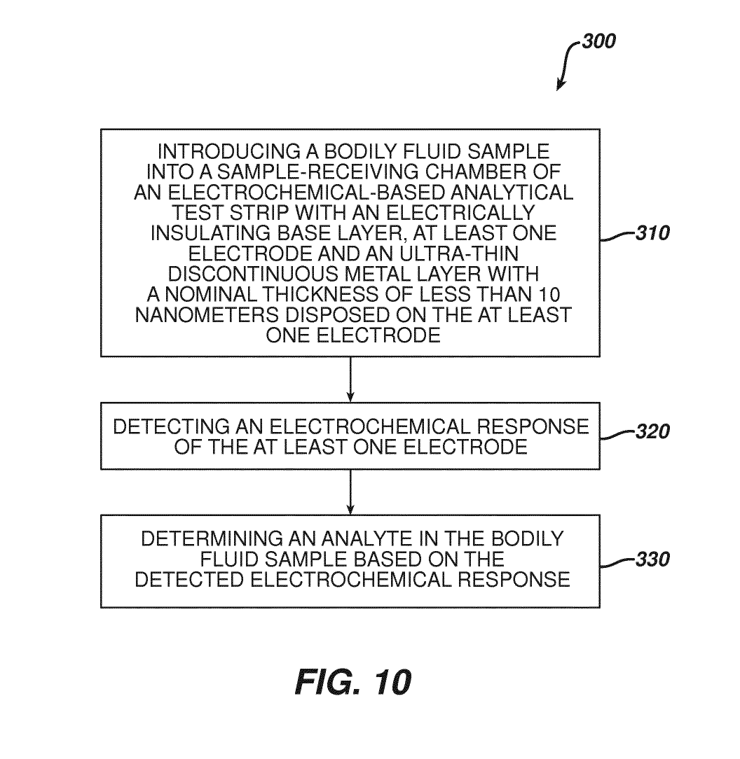

FIG. 10 is a flow diagram depicting stages in a method 300 for employing an analytical test strip according to an embodiment of the present invention. Method 300 includes, at step 310, introducing a bodily fluid sample (such as a whole blood sample) into a sample-receiving chamber of an electrochemical-based analytical test strip. In step 310, the electrochemical-based analytical test strip includes an electrically insulating base layer, at least one electrode disposed within the sample-receiving chamber and on the electrically-insulating base layer, and an ultra-thin discontinuous metal layer with a nominal thickness of less than 10 nanometers disposed above the at least one electrode and at least within the sample-receiving chamber.

Subsequently, an electrochemical response of the at least one electrode of the electrochemical-based analytical test strip is detected (see step 320 of FIG. 10). At step 330, an analyte in the bodily fluid sample is determined based on the detected electrochemical response.

Once apprised of the present disclosure, one skilled in the art will recognize that method 300 can be readily modified to incorporate any of the techniques, benefits, features and characteristics of electrochemical-based analytical test strips according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

1.-29. (canceled)

30. An electrochemical-based analytical test strip for the determination of an analyte in a bodily fluid sample, the electrochemical-based analytical test strip comprising:
an electrically insulating base layer;
a first electrically conductive layer disposed on the electrically insulating base layer and including at least one electrode;
an enzymatic reagent layer disposed on the at least one electrode;
a patterned spacer layer;
a top layer; and
an ultra-thin discontinuous metal layer with a nominal thickness of less than 10 nanometers disposed between the first electrically conductive layer and the top layer,
wherein at least the patterned spacer layer defines a sample-receiving chamber containing the at least one electrode, and
wherein the ultra-thin discontinuous metal layer is disposed at least within the sample-receiving chamber.

31. The electrochemical-based analytical test strip of claim 30 wherein the first electrically conductive layer is a carbon electrically conductive layer.

32. The electrochemically-based analytical test strip of claim 31 wherein the ultra-thin discontinuous metal layer is an ultra-thin discontinuous gold layer.

33. The electrochemical-based analytical test strip of claim 30 wherein the at least one electrode is a plurality of electrodes and the ultra-thin discontinuous metal layer is disposed on the electrically-insulating base layer and the first electrically conductive layer including at least one of the plurality of electrodes.

34. The electrochemical-based analytical test strip of claim 33 wherein the ultra-thin discontinuous metal layer is disposed on the plurality of electrodes.

35. The electrochemical-based analytical test strip of claim 33 wherein the plurality of electrodes includes a working electrode and a counter electrode and with respect to the plurality of electrodes, the ultra-thin discontinuous layer is disposed only on the counter electrode.

36. The electrochemical-based analytical test strip of claim 33 wherein the discontinuous nature of the ultra-thin discontinuous metal layer is predetermined such as to preclude an electrical path between the plurality of electrodes via the ultra-thin discontinuous metal layer.

37. The electrochemical-based analytical test strip of claim 30 further including:
a second electrically conductive layer disposed immediately below the top layer and including at least one electrode disposed in the sample-receiving chamber,
wherein the ultra-thin discontinuous metal layer is disposed on the second electrically conductive layer.

38. The electrochemically-based analytical test strip of claim 37 wherein the second electrically conductive layer includes polymer-bound graphite particles and is of free-standing mechanical integrity.

39. The electrochemical-based analytical test strip of claim 30 wherein the bodily fluid sample is a whole blood sample and the analyte is glucose.

40. The electrochemical-based analytical test strip of claim 30 wherein the nominal thickness of the ultra-thin discontinuous metal layer is in the range of 1 nano-meter to 4 nano-meters.

41. The electrochemical-based analytical test strip of claim 30 wherein the ultra-thin discontinuous metal layer has discontinuities in the range of 5 discontinuities per micron to 20 discontinuities per micron.

42. The electrochemical-based analytical test strip of claim 30 wherein the ultra-thin discontinuous metal layer is a sputter-deposited ultra-thin discontinuous metal layer.

43. The electrochemical-based analytical test strip of claim 42 wherein the sputter-deposited ultra-thin discontinuous metal layer is a sputter-deposited ultra-thin discontinuous gold layer.

44. The electrochemical-based analytical test strip of claim 42 wherein the sputter-deposited ultra-thin discontinuous metal layer is a sputter-deposited ultra-thin discontinuous metal layer is formed of at least one of palladium, platinum and silver.

45. The electrochemical-based analytical test strip of claim 42 wherein the ultra-thin discontinuous metal layer includes metal islands with a diameter no greater than 100 microns.

46. A method for employing an analytical test strip, the method comprising:
introducing a bodily fluid sample into a sample-receiving chamber of an electrochemical-based analytical test strip, the electrochemical-based analytical test strip including:
an electrically insulating base layer;
at least one electrode disposed within the sample-receiving chamber and on the electrically-insulating base layer; and
an ultra-thin discontinuous metal layer with a nominal thickness of less than 10 nanometers disposed above the at least one electrode and at least within the sample-receiving chamber;
detecting an electrochemical response of the at least one electrode of the electrochemical-based analytical test strip; and
determining an analyte in the bodily fluid sample based on the detected electrochemical response.

47. The method of claim 46 wherein the at least one electrode is a carbon electrode.

48. The method of claim 46 wherein the ultra-thin discontinuous metal layer is an ultra-thin discontinuous gold layer.

49. The method of claim 46 wherein the at least one electrode is a plurality of electrodes and the ultra-thin discontinuous metal layer is disposed on the electrically-insulating base layer and the first electrically conductive layer including at least one of the plurality of electrodes.

50. The method of claim 49 wherein the ultra-thin discontinuous metal layer is disposed on the plurality of electrodes.

51. The method of claim 49 wherein the plurality of electrodes includes a working electrode and a counter electrode and, with respect to the plurality of electrodes, the ultra-thin discontinuous layer is disposed only on the counter electrode.

52. The method of claim 49 wherein the discontinuous nature of the ultra-thin discontinuous metal layer is predetermined such as to preclude an electrical path between the plurality of electrodes via the ultra-thin discontinuous metal layer.

53. The method of claim 46 wherein the bodily fluid sample is a whole blood sample and the analyte is glucose.

54. The method of claim 46 wherein the nominal thickness of the ultra-thin discontinuous metal layer is in the range of 1 nano-meter to 4 nano-meters.

55. The method of claim 46 wherein the ultra-thin discontinuous metal layer has discontinuities in the range of 5 discontinuities per micron to 20 discontinuities per micron.

56. The method of claim 46 wherein the ultra-thin discontinuous metal layer is a sputter-deposited ultra-thin discontinuous metal layer.

57. The method of claim 56 wherein the sputter-deposited ultra-thin discontinuous metal layer is a sputter-deposited ultra-thin discontinuous gold layer.

58. The method of claim 57 wherein the sputter-deposited ultra-thin discontinuous gold layer includes gold islands with a diameter no greater than 100 microns.
